(19) United States
(12) Patent Application Publication
(10) Pub. No.: US 2024/0385336 A1
(43) Pub. Date: Nov. 21, 2024

(54) METHOD AND FILM FOR MEASURING TWO-DIMENSIONAL DISTRIBUTION OF INDUSTRIAL IRRADIATION DOSE

(71) Applicant: Zhejiang Cancer Hospital, Zhejiang (CN)

(72) Inventors: Wenjie WU, Zhejiang (CN); Guoping SHAN, Zhejiang (CN); Jinhui CHANG, Zhejiang (CN); Jiping LIU, Zhejiang (CN); Binbing WANG, Zhejiang (CN); Feng LU, Zhejiang (CN); Yixuan JIN, Zhejiang (CN); Xue BAI, Zhejiang (CN); Xiaolong CHENG, Zhejiang (CN); Xiaotong WANG, Zhejiang (CN)

(73) Assignee: Zhejiang Cancer Hospital, Zhejiang (CN)

(21) Appl. No.: 18/689,863

(22) PCT Filed: May 6, 2023
(86) PCT No.: PCT/CN2023/092619

§ 371 Date: Mar. 7, 2024

(30) Foreign Application Priority Data
May 9, 2022 (CN) 202210499681.5

(51) Int. Cl. G01T 1/20; C12Q 1/02; G01T 7/00

(52) U.S. Cl. CPC G01T 1/20; C12Q 1/02; G01T 7/005

(57) ABSTRACT

The present disclosure discloses a method for measuring two-dimensional distribution of an industrial irradiation dose based on sprouting lag time of spores, and provides a film for measuring distribution of an industrial irradiation dose. The method includes the following steps: (1) manufacturing the film: spreading a layer of spore suspension flat on a plane of a carrier, and evaporating a suspension solvent to fix a layer of spore pixel surface on the plane of the carrier; covering the carrier with a glass lid to form a sprouting cavity to cover the spore pixel surface within the sprouting cavity; and providing the glass lid with a filling hole configured to add a sprouting agent; (2) obtaining a calibration curve; and (3) measuring the irradiation dose.

### TECHNICAL FIELD

The present disclosure relates to the technical field of irradiation dose measurement, in particular to a method and film for measuring two-dimensional distribution of an industrial irradiation dose.

### BACKGROUND

An irradiation dose of industrial irradiation is generally in thousands of Gray (Gy), and a chemometric meter is commonly used as an irradiation measurement apparatus. The principle is that certain chemical substances absorb the energy of ionizing radiation and undergo a change that corresponds to the magnitude of the absorbed dose and can be measured. Common industrial irradiation measurement systems are listed in the table below.

| | | Measurement | Accuracy |
|---|---|---|---|
| Measurement System | Analytical Method | Range (Gy) | (%) |
| Fricke solution | Ultraviolet spectrophotometry | 3 × 10~4 × 10² | 1 |
| Ceric-sulfate-cerous | Ultraviolet spectrophotometry | 10³~10⁶ | 3 |
| sulfate | | | |
| Potassium dichromate | Ultraviolet-visible | 5 × 10³~4 × 10⁴ | 1 |
| | spectrophotometry | | |
| Phenethyl alcohol | Titration or Oscillography | 4 × 10²~3 × 10⁵ | 3 |
| Alanine | Electron paramagnetic | 1~10⁵ | 0.5 |
| | resonance spectroscopy | | |
| Organic glass system | Visible spectrophotometry | 10³~5 × 10⁴ | 4 |
| FWT-60 film | Visible spectrophotometry | 10³~10⁵ | 3 |
| B3/GEX film | Visible spectrophotometry | 10³~10⁵ | 3 |
| Cellulose triacetate | Ultraviolet spectrophotometry | 10⁴~10⁶ | 3 |
| Calorimetric method | Temperature | 1.5 × 10³~5 × 10⁴ | 2 |
| Lithium fluoride (Sunna | Optically Stimulated | 50~3 × 10³ | 3 |
| film) | Luminescence | | |
| Thermoluminescent | Thermoluminescence | 1~10⁴ | 2 |
| dosimeter | | | |

Current irradiation measurement devices are mainly based on physical or chemical principles, and there are also irradiation measurement methods and devices based on biological principles, such as a method for low-dose radiation bio-warning using luminescent bacteria disclosed in Chinese invention patent CN104215993A, which utilizes the sensitivity of luminescent bacteria to low-dose radiation to establish a dose-effect relationship between a radiation dose received by the bacteria and the luminescence intensity, while the luminescent intensity of the bacteria is positively correlated with the activity of luminescent elements such as luciferin, luciferase, and ATP in thalli, therefore, by calculating a luminescent intensity inhibition rate of the luminescent bacteria, the comprehensive toxicity of the low-dose radiation on the luminescent bacteria can be assessed, and the bio-warning method is established. Another example is an application of a cell cycle protein G1 (cyclin G1, CCNG1) gene as an ionizing radiation biological dosimeter as disclosed in Chinese invention patent CN103642904A. The increase in CCNG1 gene mRNA level expression in peripheral blood lymphocytes after low-dose ionizing radiation on human lymphocytoblast and after mammalian radiation is proportional to an ionizing radiation dose, and there is a certain dose-effect relationship, which can be detected quickly, easily and quantitatively by a real-time fluorescence quantitative PCR method at 24 h and 48 h after irradiation, so CCNG1 can be used as a biological dosimeter for a low-dose ionizing radiation range, and the doses of low-dose ionizing radiation to humans and mammals can be evaluated by adopting a quantitative analysis method of CCNG1 gene expression. The above irradiation dose detection using biological principles is mainly applied to low-dose measurement, and industrial irradiation measurement methods based on biological principles have not been reported.

### SUMMARY

The technical problem to be solved by embodiments of the present application is to provide a method for measuring two-dimensional distribution of an industrial irradiation dose based on sprouting lag time of spores, and provide a film for measuring distribution of an industrial irradiation dose.

In order to solve the above technical problems, the present application adopts the technical solutions described below:

A method for measuring two-dimensional distribution of an industrial irradiation dose, includes the following steps:
(1) manufacturing a film: spreading a layer of spore suspension flat on a plane of a carrier, and evaporating a suspension solvent to fix a layer of spore pixel surface on the plane of the carrier; covering the carrier with a glass lid to form a sprouting cavity to cover the spore pixel surface within the sprouting cavity; and providing the glass lid with a filling hole configured to add a sprouting agent;
(2) obtaining a calibration curve: placing the film under an irradiation device and directing irradiation of known intensity to the film with a parallel beam; and after irradiation, adding a sprouting solution containing the sprouting agent to the sprouting cavity, moving the film under a fluorescence microscope after the sprouting agent sufficiently makes contact with the spore pixel surface, monitoring fluorescence change of each spore pixel of the spore pixel surface, calculating sprouting lag time corresponding to each spore pixel and average sprouting lag time, and obtaining the calibration curve by plotting the average sprouting lag time against the corresponding irradiation intensity; and
(3) measuring the irradiation dose: placing the film under the irradiation device to receive irradiation; and after irradiation, adding the sprouting solution containing the sprouting agent to the sprouting cavity, moving the film under the fluorescence microscope after the sprouting agent sufficiently makes contact with the spore pixel surface, monitoring a fluorescence change curve of each spore pixel, calculating the sprouting lag time corresponding to each spore pixel, and inversely deducing the irradiation dose of each sprout pixel according to a plurality of known intensity calibration curves obtained in step (2), i.e., obtaining two-dimensional distribution of the industrial irradiation dose.

Further, the carrier is a tray having an agar layer, and the spore suspension is spread flat on the agar layer.

Further, the sprouting agent may be one of L-alanine, AGFK, L-valine and inosine, but not limited thereto, and the AGFK is a combination of L-asparagine, Glucose, Fructose and potassium chloride (KC1).

Further, a concentration of the sprouting agent in the sprouting solution is 1-100 mM.

Further, the sprouting solution is stored in a vessel, a rim of the vessel is provided with a pipe having a valve, and the pipe is communicated to the filling hole.

Further, the vessel is disposed on an upper portion of the glass lid and is irradiated together with the film.

Further, the irradiation dose is 10-100 kGy.

A film for measuring two-dimensional distribution of an industrial irradiation dose, includes a carrier fixedly disposed on a spore pixel surface on a plane of the carrier, and the spore pixel surface is composed of a plurality of spore pixels spread flat.

Further, the film further includes a glass lid, disposed on the carrier in a covering manner to form a sprouting cavity, and the glass lid is provided with a filling hole configured to add a sprouting agent.

Further, the carrier is a tray having an agar layer, and a spore suspension is spread flat on the agar layer.

Compared with the prior art, embodiments of the present application mainly have the following beneficial effects:

In the method for measuring two-dimensional distribution of the industrial irradiation dose based on the sprouting lag time of the spores of the present disclosure, the industrial irradiation dose is larger (more than 10 kGy), the sprouting lag time of the spores and the dose in this dose stage are in a corresponding relationship, and the irradiation dose are inversely deduced by using the lag time. The spores, the carrier and the sprouting agent solution needed for manufacturing the film based on the spore pixel surface are easy to obtain and are relatively inexpensive, can be purchased directly from the market, are low in production cost, and can be produced in large quantities as disposable supplies. The method is easy to operate, and has low cost, good accuracy and low environmental risk. According to the present disclosure, measurement of the two-dimensional distribution of the industrial irradiation dose can be performed by the spore pixel surface, so as to achieve measurement of dose information of the whole irradiation surface, and is more targeted compared with traditional fixed-point measurement of dose information of an irradiation center.

### BRIEF DESCRIPTION OF FIGURES

FIG. 1 is a relation diagram of sprouting lag time of spores after irradiation and fluorescence intensity according to the present disclosure; and

FIG. 2 is a structural decomposition schematic diagram of a film according to the present disclosure.
In the figures, 1—tray, 2—spore pixel surface, 3—spore, 4—glass lid, 6—filling hole, 7—sprouting solution, 8—vessel, and 9—pipe.

### DETAILED DESCRIPTION

Unless otherwise defined, all technical and scientific terms used herein have the same meaning as commonly understood by those skilled in the art of the present application; the terms used herein in the specification of the application are used only for the purpose of describing specific embodiments, and are not intended to limit the present application; and the terms “including” and “having” and any variations thereof, as used in the specification of the present application and claims, are intended to cover non-exclusive inclusion. The terms “first”, “second”, etc. in the specification and claims of the present application or the accompanying drawings described above are used to distinguish different objects and are not intended to describe a particular order.

Reference to “embodiments” herein implies that particular features, structures, or characteristics described in conjunction with embodiments may be included in at least one embodiment of the present application. The presence of the phrase at various positions in the specification does not necessarily refer to the same embodiment, nor is it a separate or alternative embodiment that is mutually exclusive of other embodiments. It is understood by those skilled in the art, both explicitly and implicitly, that the embodiments described herein may be combined with other embodiments.

The present disclosure provides a method for measuring two-dimensional distribution of an industrial irradiation dose, including the following steps:
(1) A film is manufactured: a layer of spore suspension is spread flat on a plane of a carrier, and a suspension solvent is evaporated to fix a layer of spore pixel surface on the plane of the carrier; the carrier is covered with a glass lid to form a sprouting cavity to cover the spore pixel surface within the sprouting cavity; and the glass lid is provided with a filling hole configured to add a sprouting agent.
Before the suspension is spread flat, the suspension needs to be sufficiently mixed (either by shaking, ultrasonic mixing, or a combination of both) to avoid caking of spores. The suspension is sufficiently mixed well and then spread flat to ensure that the spores can be distributed more evenly and randomly on the plane of the carrier. Preferably, the spores are evenly and closely distributed on the plane of the carrier in a monolayer manner. If the spores are stacked, fluorescent spots cannot be observed (only a large overlapping fluorescence can be observed), and if the spores are too sparse, a dose of a small number of spots in the plane can only be measured, which affects the detection effect.
(2) A calibration curve is obtained: the film is placed under an irradiation device and irradiation of known intensity is directed to the film with a parallel beam, wherein the parallel beam may be understood as the irradiation intensity being the same everywhere in the cross section of the irradiation beam; and after irradiation, a sprouting solution containing the sprouting agent is added to the sprouting cavity, the film is moved under a fluorescence microscope after the sprouting agent sufficiently makes contact with the spore pixel surface, fluorescence change of each spore pixel of the spore pixel surface is monitored, sprouting lag time corresponding to each spore pixel and an average sprouting lag time are calculated, and the calibration curve is obtained by plotting the average sprouting lag time against the corresponding irradiation intensity, wherein the known intensity of the above irradiation may include, but is not limited to, 10 kGy, 20 kGy, 30 kGy, 40 kGy, 50 kGy, 60 kGy, 70 kGy, 80 kGy, 90 kGy, 100 kGy, and one known irradiation intensity corresponds to one calibration curve.
(3) The irradiation dose is measured: the film is placed under the irradiation device to receive irradiation, wherein intensities at all parts of the cross section of an actual irradiation beam are inconsistent; and after irradiation, the sprouting solution containing the sprouting agent is added to the sprouting cavity, the film is moved under the fluorescence microscope after the sprouting agent sufficiently makes contact with the spore pixel surface, a fluorescence change curve of each spore pixel is monitored, the sprouting lag time corresponding to each spore pixel is calculated, and the irradiation dose of each sprout pixel is inversely deduced according to a plurality of known intensity calibration curves obtained in step (2), i.e., two-dimensional distribution of the industrial irradiation dose is obtained.

Some spore-producing bacteria will produce spores under adverse environmental conditions (e.g., extreme lack of nutrients). Spores are a special kind of dormant body of bacteria, have no metabolism inside, stable nature and very strong resistance to harsh environment, and are not likely to die. In the right conditions (such as supply of nutrients is restored, and the environment is suitable for growth), the spores can quickly sprout, and ultimately grow into thalli. The spores internally contain a large amount of dipicolinic acid (DPA), and in the germination, DPA is released into the surrounding environment. If Tb³⁺ is present in the external environment, the released DPA chelates with Tb³⁺ to form the highly fluorescent Tb-DPA. Under suitable excitation light (an excitation wavelength of about 278 nm), an emission spectrum of Tb-DPA has significant peaks in the range of 532 nm to 557 nm, and the intensity of the peaks is directly proportional to the content of DPA. Thus the sprouting process of the spores can be monitored indirectly by monitoring the enhancement of the fluorescence intensity with time.

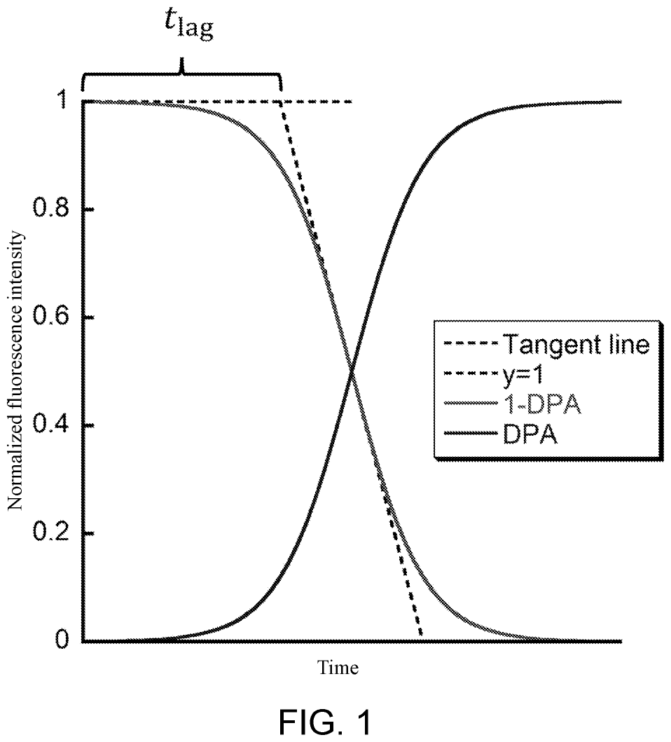

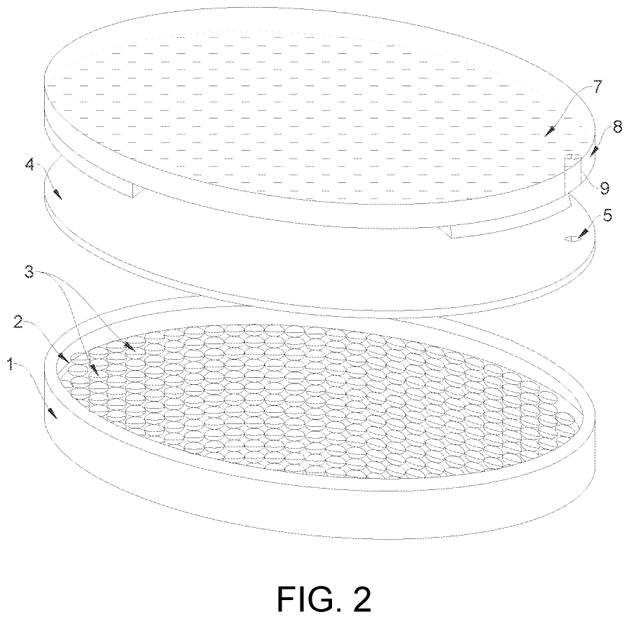

The spores do not release DPA immediately upon sprouting, but have a period of lag time (tlag), as shown in FIG. 1. The fluorescence curve at the time of sprouting is observed by the fluorescence microscope, and corresponds to the DPA release process (-DPA). If data points are discrete, the data points are fitted by a Sigmoid function. A 1-DPA curve (-1-DPA) is drawn. A tangent line ( [symbol] Tangent line) is made at the point where the slope of the 1-DPA curve is the highest, and the intercept of the tangent line at y=1 ( [symbol] y=1) is the tlag.

The inventor found that, in the range of thousands of Gy, the more irradiation dose (D) the spores are subjected to, the longer the sprouting lag time is, and the irradiation dose and the sprouting lag time are in a corresponding relationship, which is mainly due to some Germinant Receptors on the inner membranes of the spores being damaged by irradiation, which makes the response of the spores to the sprouting agent become slow. Thus, the inventor can use the sprouting lag time of the spores to inversely deduce the irradiation dose to which the spores are exposed, and measure the industrial irradiation dose on the basis of this principle. On the other hand, dried spores are stable in property and easy to store for a long period of time, and the spores are non-pathogenic and non-biohazardous.

In the method for measuring two-dimensional distribution of the industrial irradiation dose based on the sprouting lag time of the spores of the present disclosure, the industrial irradiation dose is larger (more than 10 kGy), the sprouting lag time of the spores and the dose in this dose stage are in a corresponding relationship, and the irradiation dose are inversely deduced by using the lag time. The spores, the carrier and the sprouting agent solution needed for manufacturing the film based on the spore pixel surface are easy to obtain and are relatively inexpensive, can be purchased directly from the market, are low in production cost, and can be produced in large quantities as disposable supplies. The method is easy to operate, and has low cost, good accuracy and low environmental risk. According to the present disclosure, measurement of the two-dimensional distribution of the industrial irradiation dose can be performed by the spore pixel surface, so as to achieve measurement of dose information of the whole irradiation surface, and is more targeted compared with traditional fixed-point measurement of dose information of an irradiation center.

The present disclosure is described in detail below in combination with embodiments, which are only preferred embodiments of the present disclosure and are not a limitation to the present disclosure.

### Embodiment 1

The present disclosure provides a method for measuring two-dimensional distribution of an industrial irradiation dose, including the following steps:
(1) a film is manufactured: a rectangular tray is provided as a carrier, hot agar (containing 1.5% agar and 100 μM TbC13) is poured, the hot agar is left to solidify to form an agar layer, a layer of spore suspension is spread flat on the agar layer, and a suspension solvent is evaporated to fix a layer of spore pixel surface, which consists of a plurality of spore pixels, on the plane of the agar layer;
the tray is covered with a glass lid to form a sprouting cavity to cover the spore pixel surface within the sprouting cavity; and the glass lid is provided with a filling hole configured to add a sprouting agent;
(2) a calibration curve is obtained: the film is placed under an irradiation device and irradiation of 10 kGy intensity is directed to the film with a parallel beam, wherein the parallel beam may be understood as the irradiation intensity being the same everywhere in the cross section of the irradiation beam; and after irradiation, a sprouting solution containing 20 mM L-alanine as the sprouting agent is added to the sprouting cavity, the film is moved under a fluorescence microscope after the sprouting agent sufficiently makes contact with the spore pixel surface, fluorescence change of each spore pixel of the spore pixel surface is monitored, sprouting lag time corresponding to each spore pixel and average sprouting lag time are calculated, and a 10 kGy calibration curve is obtained by plotting the average sprouting lag time against the corresponding irradiation intensity; and a 20 kGy calibration curve, a 30 kGy calibration curve, a 40 kGy calibration curve, a 50 kGy calibration curve, a 60 kGy calibration curve, a 70 kGy calibration curve, an 80 kGy calibration curve, a 90 kGy calibration curve, and a 100 kGy calibration curve are obtained by means of the same method; and
(3) the irradiation dose is measured: the film is placed under the irradiation device to receive irradiation; and after irradiation, the sprouting solution containing the 20 mM L-alanine as the sprouting agent is added to the sprouting cavity, the film is moved under the fluorescence microscope after the sprouting agent sufficiently makes contact with the spore pixel surface, a fluorescence change curve of each spore pixel is monitored, the sprouting lag time corresponding to each spore pixel is calculated, and the irradiation dose of each sprout pixel is inversely deduced according to a plurality of known intensity calibration curves obtained in step (2), i.e., two-dimensional distribution of the industrial irradiation dose is obtained.

### Embodiment 2

The present disclosure provides a method for measuring two-dimensional distribution of an industrial irradiation dose, including the following steps:
(1) a film is manufactured: an oval tray is provided as a carrier, hot agar (containing 1.5% agar and 100 μM TbC13) is poured, the hot agar is left to solidify to form an agar layer, a layer of spore suspension is spread flat on the agar layer, and a suspension solvent is evaporated to fix a layer of spore pixel surface, which consists of a plurality of spore pixels, on the plane of the agar layer;
the tray is covered with a glass lid to form a sprouting cavity to cover the spore pixel surface within the sprouting cavity; and the glass lid is provided with a filling hole configured to add a sprouting agent;
(2) a calibration curve is obtained: the film is placed under an irradiation device and irradiation of 10 kGy intensity is directed to the film with a parallel beam, wherein the parallel beam may be understood as the irradiation intensity being the same everywhere in the cross section of the irradiation beam; and after irradiation, a sprouting solution containing the sprouting agent (asparagine, Glucose, Fructose and potassium chloride, 50 mM respectively) is added to the sprouting cavity, the film is moved under a fluorescence microscope after the sprouting agent sufficiently makes contact with the spore pixel surface, fluorescence change of each spore pixel of the spore pixel surface is monitored, sprouting lag time corresponding to each spore pixel and average sprouting lag time are calculated, and a 10 kGy calibration curve is obtained by plotting the average sprouting lag time against the corresponding irradiation intensity; and a 15 kGy calibration curve, a 20 kGy calibration curve, a 25 kGy calibration curve, a 30 kGy calibration curve, a 35 kGy calibration curve, a 40 kGy calibration curve, a 45 kGy calibration curve, a 50 kGy calibration curve, and a 60 kGy calibration curve are obtained by means of the same method; and
(3) the irradiation dose is measured: the film is placed under the irradiation device to receive irradiation; and after irradiation, the sprouting solution containing the sprouting agent (asparagine, Glucose, Fructose and potassium chloride, 50 mM respectively) is added to the sprouting cavity, the film is moved under the fluorescence microscope after the sprouting agent sufficiently makes contact with the spore pixel surface, a fluorescence change curve of each spore pixel is monitored, the sprouting lag time corresponding to each spore pixel is calculated, and the irradiation dose of each sprout pixel is inversely deduced according to a plurality of known intensity calibration curves obtained in step (2), i.e., two-dimensional distribution of the industrial irradiation dose is obtained.

### Embodiment 3

The present disclosure provides a method for measuring two-dimensional distribution of an industrial irradiation dose, including the following steps:
(1) a film is manufactured: a round tray is provided as a carrier, hot agar (containing 1.5% agar and 100 μM TbC13) is poured, the hot agar is left to solidify to form an agar layer, a layer of spore suspension is spread flat on the agar layer, and a suspension solvent is evaporated to fix a layer of spore pixel surface, which consists of a plurality of spore pixels, on the plane of the agar layer;
the tray is covered with a glass lid to form a sprouting cavity to cover the spore pixel surface within the sprouting cavity; and the glass lid is provided with a filling hole configured to add a sprouting agent;
a sprouting solution containing 15 mM L-valine as the sprouting agent is stored in a vessel, wherein a rim of the vessel is provided with a pipe having a valve, the pipe is communicated to the filling hole, and the vessel is disposed on an upper portion of the glass lid as shown in FIG. 2;
(2) a calibration curve is obtained: the film and the vessel are placed under an irradiation device and irradiation of 50 kGy intensity is directed to the film with a parallel beam, wherein the parallel beam may be understood as the irradiation intensity being the same everywhere in the cross section of the irradiation beam; and after irradiation, a valve is opened to add a sprouting solution to the sprouting cavity through the pipe, the film is moved under a fluorescence microscope after the sprouting agent sufficiently makes contact with the spore pixel surface, fluorescence change of each spore pixel of the spore pixel surface is monitored, sprouting lag time corresponding to each spore pixel and average sprouting lag time are calculated, and a 50 kGy calibration curve is obtained by plotting the average sprouting lag time against the corresponding irradiation intensity; and a 55 kGy calibration curve, a 60 kGy calibration curve, a 65 kGy calibration curve, a 70 kGy calibration curve, a 75 kGy calibration curve, an 80 kGy calibration curve, an 85 kGy calibration curve, a 90 kGy calibration curve, a 95 kGy calibration curve, and a 100 kGy calibration curve are obtained by means of the same method; and
(3) the irradiation dose is measured: the film is placed under the irradiation device to receive irradiation; and after irradiation, the valve is opened to add the sprouting solution to the sprouting cavity through the pipe, the vessel and the pipe are moved away first and then the film is moved under the fluorescence microscope after the sprouting agent sufficiently makes contact with the spore pixel surface, a fluorescence change curve of each spore pixel is monitored, the sprouting lag time corresponding to each spore pixel is calculated, and the irradiation dose of each sprout pixel is inversely deduced according to a plurality of known intensity calibration curves obtained in step (2), i.e., two-dimensional distribution of the industrial irradiation dose is obtained.

Obviously, the above-described embodiments are only a part of the embodiments of the present application and not all of the embodiments, and the accompanying drawings give a preferred embodiment of the present application without limiting the patent scope of the present application. The present application may be implemented in many different forms, and conversely, these embodiments are provided for the purpose of making the understanding of the disclosed content of the present application more thorough and comprehensive. Although the present application has been described in detail with reference to the foregoing embodiments, it is still possible for a person skilled in the art to modify the technical solutions recorded in the foregoing specific embodiments or to make equivalent substitutions for some of the technical features therein. Any equivalent structure made by utilizing the contents of the specification of the present application and the accompanying drawings, which is directly or indirectly applied in other related technical fields, is similarly within the scope of the patent protection of the present application.

1. A method for measuring two-dimensional distribution of an industrial irradiation dose, comprising the following steps:
(1) manufacturing a film: spreading a layer of spore suspension flat on a plane of a carrier, and evaporating a suspension solvent to fix a layer of spore pixel surface on the plane of the carrier; covering the carrier with a glass lid to form a sprouting cavity to cover the spore pixel surface within the sprouting cavity; and providing the glass lid with a filling hole configured to add a sprouting agent;
(2) obtaining a calibration curve: placing the film under an irradiation device and directing irradiation of known intensity to the film with a parallel beam; and after irradiation, adding a sprouting solution containing the sprouting agent to the sprouting cavity, moving the film under a fluorescence microscope after the sprouting agent sufficiently makes contact with the spore pixel surface, monitoring fluorescence change of each spore pixel of the spore pixel surface, calculating sprouting lag time corresponding to each spore pixel and average sprouting lag time, and obtaining the calibration curve by plotting the average sprouting lag time of each sprout pixel point against the corresponding irradiation intensity; and
(3) measuring the irradiation dose: placing the film under the irradiation device to receive irradiation; and after irradiation, adding the sprouting solution containing the sprouting agent to the sprouting cavity, moving the film under the fluorescence microscope after the sprouting agent sufficiently makes contact with the spore pixel surface, monitoring a fluorescence change curve of each spore pixel, calculating the sprouting lag time corresponding to each spore pixel, and inversely deducing the irradiation dose of each sprout pixel according to a plurality of known intensity calibration curves obtained in step (2), so as to obtain two-dimensional distribution of the industrial irradiation dose.

2. The method for measuring two-dimensional distribution of the industrial irradiation dose according to claim 1, wherein the carrier is a tray having an agar layer, and the spore suspension is spread flat on the agar layer.

3. The method for measuring two-dimensional distribution of the industrial irradiation dose according to claim 1, wherein the sprouting agent is one of L-alanine, AGFK, L-valine and inosine.

4. The method for measuring two-dimensional distribution of the industrial irradiation dose according to claim 1, wherein a concentration of the sprouting agent in the sprouting solution is 1-100 mM.

5. The method for measuring two-dimensional distribution of the industrial irradiation dose according to claim 1, wherein the sprouting solution is stored in a vessel, a rim of the vessel is provided with a pipe having a valve, and the pipe is communicated to the filling hole.

6. The method for measuring two-dimensional distribution of the industrial irradiation dose according to claim 5, wherein the vessel is disposed on an upper portion of the glass lid and is irradiated together with the film.

7. The method for measuring two-dimensional distribution of the industrial irradiation dose according to claim 1, wherein the irradiation dose is 10-100 kGy.

8. A film for measuring two-dimensional distribution of an industrial irradiation dose, characterized in that, comprising a carrier fixedly disposed on a spore pixel surface on a plane of the carrier, wherein the spore pixel surface is composed of a plurality of spore pixels spread flat.

9. The film for measuring two-dimensional distribution of the industrial irradiation dose according to claim 8, wherein the film further comprises a glass lid, disposed on the carrier in a covering manner to form a sprouting cavity, and the glass lid is provided with a filling hole configured to add a sprouting agent.

10. The film for measuring two-dimensional distribution of the industrial irradiation dose according to claim 8, wherein the carrier is a tray having an agar layer, and a spore suspension is spread flat on the agar layer.
